(19) United States
(12) Patent Application Publication
(10) Pub. No.: US 2020/0069191 A1
(43) Pub. Date: Mar. 5, 2020

(54) TEMPERATURE-BASED DENSITY-AGNOSTIC SYSTEM AND METHOD FOR BREAST CANCER DIAGNOSIS

(71) Applicant: Higia, Inc., Wilmington New Castle, DE (US)

(72) Inventors: Julián Ríos Cantú, Monterrey (MX); José Antonio Torres González, Monterrey (MX); Raymundo González Leal, Monterrey (MX); Juan Carlos López Pendas, Puebla (MX)

(21) Appl. No.: 16/534,993

(22) Filed: Aug. 7, 2019

Related U.S. Application Data
(60) Provisional application No. 62/715,743, filed on Aug. 7, 2018.

(51) Int. Cl. A61B 5/01; A61B 5/00

(52) U.S. Cl. CPC A61B 5/01; A41C 3/0064; A61B 5/4312

(57) ABSTRACT

A system for evaluating breast cancer risk in a user includes: a temperature-sensing brassiere including: a first sensor mesh comprising: a first flexible mesh defining a mesh pattern and extending across a first breast region of the user; a first set of temperature-sensing assemblies arranged at intersections in the first flexible mesh; and for each temperature-sensing assembly in the first set of temperature-sensing assemblies, an electrical trace in the first mesh electrically coupling the temperature-sensing assembly to a controller; a brassiere structure configured to locate the first sensor mesh across the first breast region of the user; and the controller configured to: sample each temperature-sensing assembly in the first set of temperature-sensing assemblies during a data collection period to generate a series of temperature data; and store the series of temperature data.

### CROSS-REFERENCE TO RELATED APPLICATIONS

This Application claims the benefit of U.S. Provisional Application No. 62/715,743, filed on 7 Aug. 2018, which is incorporated in its entirety by this reference.

### TECHNICAL FIELD

This invention relates generally to the field of medical diagnostic equipment and more specifically to a new and useful system and method for breast health monitoring in the field of medical diagnostic equipment.

### BACKGROUND

Although mammography is the current standard of care in breast cancer screening and detection, it has many limitations. For example, mammography is ineffective in detecting breast cancer in women with dense breast tissue, as fibroglandular tissue obscures tumors in the mammography image. A study of 329,495 women found that the sensitivity of mammography varies between 62.9% and 69.4% for women with dense breasts, compared to a sensitivity of between 81.5% and 87% for women with predominantly fatty breasts (Patricia A. Carney et al., Annals of Internal Medicine. 2003; 138: 168-175). 40% of women in the United States and nearly 80% of women of Asian descent have dense breast tissue (Jeffrey A. Tice et al., Annals of Internal Medicine. 2008; 148: 337-347). Additionally, women with dense breast tissue are two to six times more likely to develop breast cancer (Richard J. Santen et al., New England Journal of Medicine. 2005; 353: 275-285).

Breast cancer screening for young women is also neglected in the current standard of care. Mammograms are not recommended for young women due to concerns related to radiation exposure and the frequency of false positives associated with denser breasts. Yet, 7% of breast cancer cases involve women younger than 40 years of age and World Health Organization predicts that the incidence of breast cancer in young women will increase by 50% by the year 2035.

Although alternative methods for detecting breast cancer, such as thermography, have previously been explored, these methods lack sensitivity when compared to a mammogram and are not typically recommended by medical professionals. Additionally, thermographic methods utilizing thermal imaging have inherent problems in accurately detecting temperature and preventing measurement drift. For example, thermal cameras detect the intensity of infrared light emanating from a target surface, which can include reflected, transmitted, or emitted infrared light. A thermal imaging camera uses a number of algorithms to determine the temperature of a surface, thereby increasing uncertainty in the temperature measurement. Furthermore, thermal cameras with the accuracy necessary for thermography are prohibitively expensive to many healthcare providers.

### BRIEF DESCRIPTION OF THE FIGURES

FIG. 1 is a schematic representation of a system;

FIG. 2 is a schematic representation of the system;

FIGS. 3A, 3B, and 3C are schematic representations of the system;

FIGS. 4A, 4B, and 4C are schematic representations of the system; and

FIG. 5 is a flowchart representation of a method; and

FIG. 6 is a flowchart representation of a method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. System

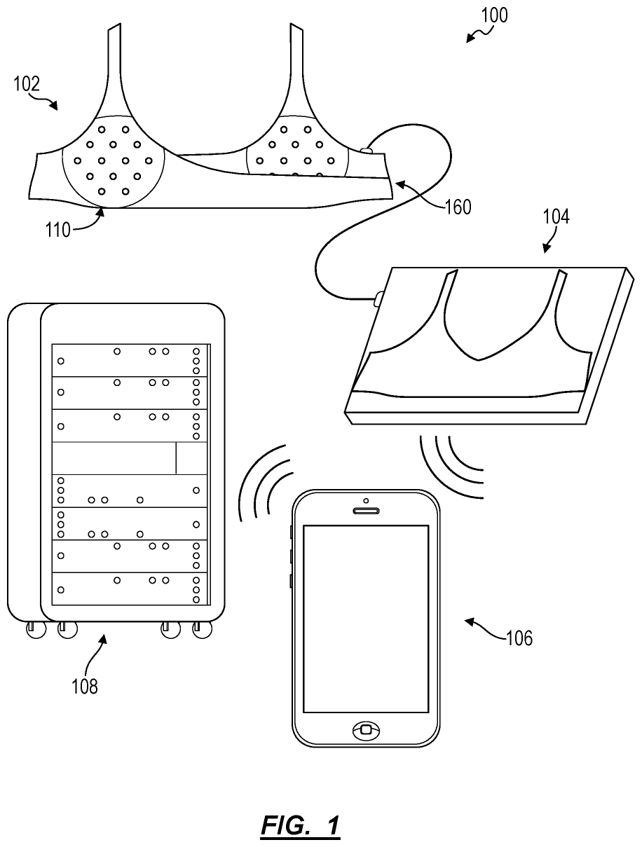

As shown in FIG. 1, a system 100 for evaluating breast cancer risk in a user includes, a temperature-sensing brassiere 102 including a first sensor mesh 110, a brassiere structure 160, and a controller 170. The first sensor mesh 110 includes: a first flexible mesh 120 defining a mesh pattern and extending across a first breast region of the user; a first set of temperature-sensing assemblies 130 arranged at intersections in the first flexible mesh 120; and for each temperature-sensing assembly in the first set of temperature-sensing assemblies, an electrical trace 140 in the first mesh electrically coupling the temperature-sensing assembly to a controller 150. The temperature-sensing brassiere also includes the brassiere structure 160 that is configured to locate the first sensor mesh 110 across the first breast region of the user. The temperature-sensing brassiere further includes the controller 150 configured to: sample each temperature-sensing assembly in the first set of temperature-sensing assemblies during a data collection period to generate a series of temperature data; and store the series of temperature data.

One variation of the system 100 for evaluating breast cancer risk in a user includes a temperature-sensing brassiere 102, including: a first substrate 122, a first set of sensing assemblies 130, a brassiere structure 160, and a controller 170. This variation of the system includes the first substrate defining a mesh pattern and including: a first set of boustrophedonic segments 124 arranged in the mesh pattern; and a first set of island regions 126 at each intersection of the first set of boustrophedonic segments 124 in the mesh pattern. This variation of the system 100 also includes the first set of sensing assemblies 130, each sensing assembly 130 in the first set of sensing assemblies arranged at an island region 126 in the first set of island regions 126 and including: a thermal contact 132 fastened to the first substrate 122 at the island region 126 via a bore 128 in the first substrate 122 at the island region 126; and a temperature sensor 134 coupled to the first substrate at the island region 126 and configured to detect the temperature of the thermal contact 132. This variation of the system 100 also includes the brassiere structure 160: configured to locate the first substrate 122 over a first breast region of the user; and interposed between the first substrate 122 and the thermal contact 132 of each sensing assembly 130 in the first set of sensing assemblies 130, wherein the thermal contact 132 of each sensing assembly 130 in the first set of sensing assemblies 130 protrudes through the first substrate 122 and the brassier structure 160 to contact skin of the user proximal the first breast region of the user. This variation of the system 100 also includes a controller 170 electrically coupled to the set of temperature sensors 134 via the first substrate 122 and configured to sample the temperature sensor 134 of each sensing assembly 130 in the first set of sensing assemblies 130 during a data collection period to generate a series of temperature data and store the series of temperature data.

### 2. Method

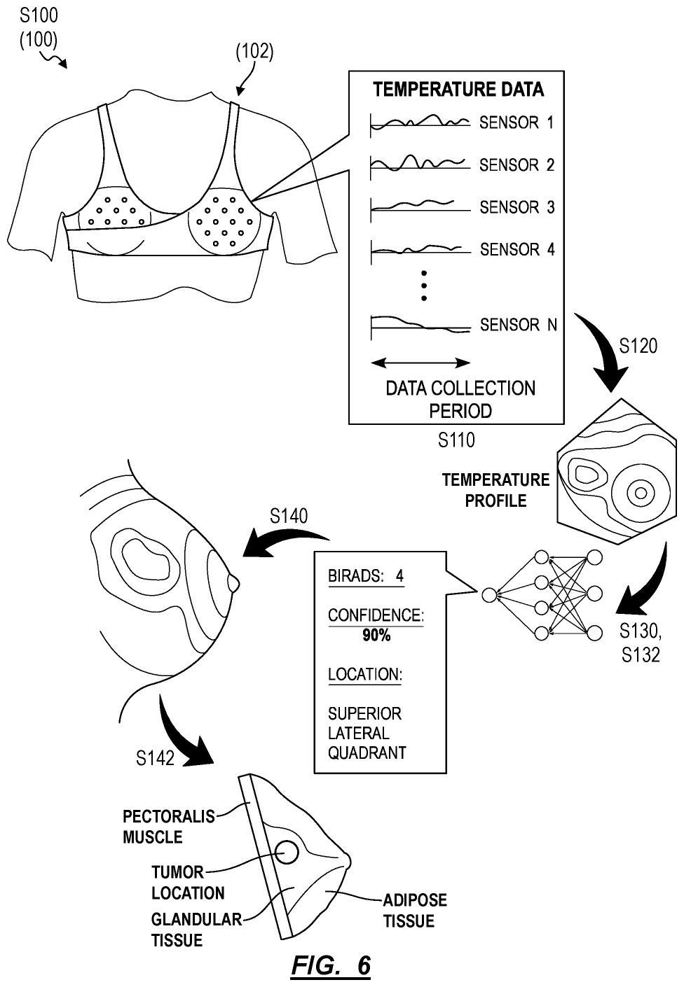

As shown in FIG. 6, a method S100 for estimating a breast cancer risk assessment includes, at the temperature-sensing brassiere 102, during a data collection period, recording first breast temperature data from each temperature-sensing assembly 130 in the first set of temperature-sensing assemblies 130 and second breast temperature data from the second set of temperature-sensing assemblies 130 in Block S110. The method S100 also includes, after the data collection period: spatially interpolating the first breast temperature data and the second breast temperature data to generate a first breast temperature profile and a second breast temperature profile in Block S120; and estimating a breast cancer risk assessment for the user based on the first breast temperature data and the second breast temperature data in Block S130.

One variation of the method S100 includes rendering the first breast temperature profile and the second breast temperature profile as a heat map representing the temperature of the first breast in Block S140.

Another variation of the method S100 also includes: estimating a location of an abnormal mass in the first breast of the user or the second breast of the user in Block S132; and rendering an indication of the location of the abnormal mass in the first breast temperature profile or the second breast temperature profile in Block S142.

### 3. Applications

Generally, the method S100 for estimating a breast cancer risk assessment is executed by a system 100 including a temperature-sensing brassiere 102 connected to a computational device 106, which can be a desktop computer, laptop computer, smartphone, or any other computational device 106 capable of a wired or wireless connection with the temperature-sensing brassiere 102. The temperature-sensing brassiere 102 records temperature data, and optionally inertial measurement data, from the surface of each of a user's breasts during a data collection period. The computational device 106 executes software that can control the temperature-sensing brassiere 102 and perform analysis on the data collected from the temperature-sensing brassiere 102 in order to estimate a breast cancer risk assessment for the user. The computational device 106 can execute the software locally or remotely over a network such as the Internet.

In particular, the temperature-sensing brassiere 102 includes a brassiere structure that maximizes adjustability through the use of elastic fabrics, such as an adjustable sports brassiere. However, the temperature-sensing brassiere 102 includes left and right sensor meshes 110 instead of cups in order to detect the skin temperature across the breast region of the user.

In one implementation, the sensor mesh 110 includes a substrate 122 such as a flexible printed circuit board (hereinafter “PCB”) that defines a mesh pattern. The substrate 122 includes a set of flexible segments arranged in the mesh pattern—such as boustrophedonic segments 124—that are characterized by a shape that enables the flexible segments to increase in length while under tension. At each intersection of the set of flexible segments, the substrate 122 includes an island region 126, which can be circular in shape and functions as a point of attachment for sensing assemblies of the temperature-sensing brassiere 102. Thus, the substrate 122, supported by the brassiere structure, can stretch across the surface of a breast of the user, thereby evenly distributing temperature-sensing assemblies 130 across the breast of the user while imparting even pressure on the breast of the user so as not to deform the natural shape of the breast or cause changes in blood circulation within the breast of the user.

Each temperature-sensing assembly 130 includes a temperature sensor 134 (e.g., an integrated circuit arranged on flexible PCB substrate 122), which is electrically coupled to a controller 150 of the temperature-sensing brassiere 102 via electrical trace 140(s) printed directly onto the substrate 122 (and can include a low and high voltage source, serial data channel, and/or a serial clock channel). Thus, the temperature-sensing brassiere 102 can repeatedly sample each temperature sensor 134 distributed across the surface of a user's breast via the flexible substrate 122.

Additionally, the substrate 122 can be printed as a single component simplifying the manufacturing process of the temperature-sensing brassiere 102. The temperature-sensing brassiere 102 can include two substrate 122s (one for each of the user's breasts) that each extend from the sternum to the axillary region (i.e. underarm region) and from below the breast to the clavicular region of the user when the temperature-sensing brassiere 102 is worn by the user.

In another implementation, each sensor mesh 110 includes an elastic fabric mesh that stretches to accommodate and conform to the breast of a user wearing the temperature-sensing brassiere 102. As such, the elastic fabric mesh extends from the sternum to the axillary region and from below the breast to the clavicular region. A set of temperature sensors 134 are arranged at the intersections in the elastic fabric mesh, such that when the elastic fabric mesh stretches to accommodate a breast of the user, the temperature sensors 134 are distributed across the surface of the user's breast. Each of the temperature sensors 134 is connected, via an elastic wire 142 imbedded in the elastic fabric mesh, to a controller 150 in the temperature-sensing brassiere 102.

Before executing the method S100, a user can be admitted to a temperature-controlled environment (e.g. an examination room) and is asked to disrobe from the waist up. This allows the surface temperature of the user's breasts to reach a temperature equilibrium before the test begins. In some cases, the temperature-controlled environment can be quite cold in order to “cold shock” the breasts of the user. This reduces blood flow in the user's breasts, such that, when some form of insulation from the temperature-controlled environment is applied to the user's breasts, blood will flow back into the breasts, thereby magnifying the effect of angiogenic regions in the breast potentially caused by a cancerous growth.

The temperature-sensing brassiere 102 is then secured to the user, such that the sensing assemblies of the temperature-sensing brassiere 102 are distributed across breast regions of the user. The method S100 can also include recording the location of each of the user's nipples relative to the sensor mesh 110. For example, the method S100 can include recording an identification or relative location of a temperature sensor 134 nearest to the left and the right nipple of the user. The method S100 can then compensate for any differences in the positioning of the left and right breasts of the user relative to the sensor mesh 110. Alternatively, the method S100 can automatically estimate the location of the user's nipples based on the series of temperature data collected at the temperature-sensing brassiere 102.

The system 100 can initiate a data collection period once the user has secured the temperature-sensing brassiere 102 to her breasts. During the data collection period, the system 100 records temperature data from each temperature sensor 134 in each sensing assembly in the temperature-sensing brassiere 102 via a wireless or wired data stream, or by storing the temperature data directly at the temperature-sensing brassiere 102. The temperature-sensing brassiere 102 can store temperature data in association with the sensor from which they were sampled and with a timestamp indicating the time elapsed since the beginning of the data collection period.

Upon completion of a data collection period, the user can remove the temperature-sensing brassiere 102. The controller 150 can then transmit the collected temperature data to another computational device 106 for further processing or, alternatively, perform post processing at the temperature-sensing brassiere 102. In one implementation, the system 100 also includes a hub 104, which can extract the recorded temperature data from the temperature-sensing brassiere 102 and includes a wireless communication chip in order to transmit the data to an internet connected computational device 106. The computational device 106 can then transmit the temperature data to remote server 108s of the system 100, which can execute the breast cancer risk assessment model and estimate a breast cancer risk assessment for the user based on the temperature data from the temperature-sensing brassiere 102.

In order to estimate a breast cancer risk assessment for the user, the system 100 can spatially normalize the temperature data to correct for differences in size and orientation of each breast. Additionally, in one implementation, the system 100 can interpolate between the locations of each sensor in the sensor mesh 110 to generate a two-dimensional or three-dimensional temperature profile of the breasts of a user over the data collection period. In one variation, the temperature-sensing brassiere 102 includes inertial measurement units (hereinafter IMUs) arranged across the sensor mesh 110 to estimate the size, shape, and mass of each breast of the user. The method S100 can include incorporating the IMU data when calculating the three-dimensional temperature profile of the user's breasts.

After the system 100 collects temperature data for the breasts of the user, the system 100 can execute a machine learning model based on the temperature data in order to detect temperature anomalies in the breast tissue of the user. These temperature anomalies may be caused by angiogenesis within the breast or other effects that cancerous growths may have on the surrounding breast tissue. By leveraging the temperature data from the user's breasts, the machine learning model can identify, characterize, and locate cancerous growths, pre-cancerous growths, or any other cause of temperature anomalies in the breast tissue, such as hormone induced temperature changes, non-cancerous growths, circadian temperature variation, etc.

In one implementation, the system 100 can display the three-dimensional temperature profile of the user's breasts as a three-dimensional heat map to medical professionals for subjective analysis and corroboration of the machine learning model.

The method S100 improves upon previous technology by precisely distributing the temperature sensors 134 over a variety of breast sizes and shapes using the temperature-sensing brassiere 102, by providing more accurate temperature data than thermographic imagery (i.e. to within 0.1 degrees Celsius), and by utilizing a thermodynamic model for heat transfer within the breast to create a three-dimensional temperature profile based on surface temperature data. The method S100 also collects a much greater volume of data when compared to standard thermography and thereby enabling application of a machine learning model, as opposed to a visual analysis performed by a medical professional, to assess cancer risk.

### 4. Temperature-Sensing Brassiere

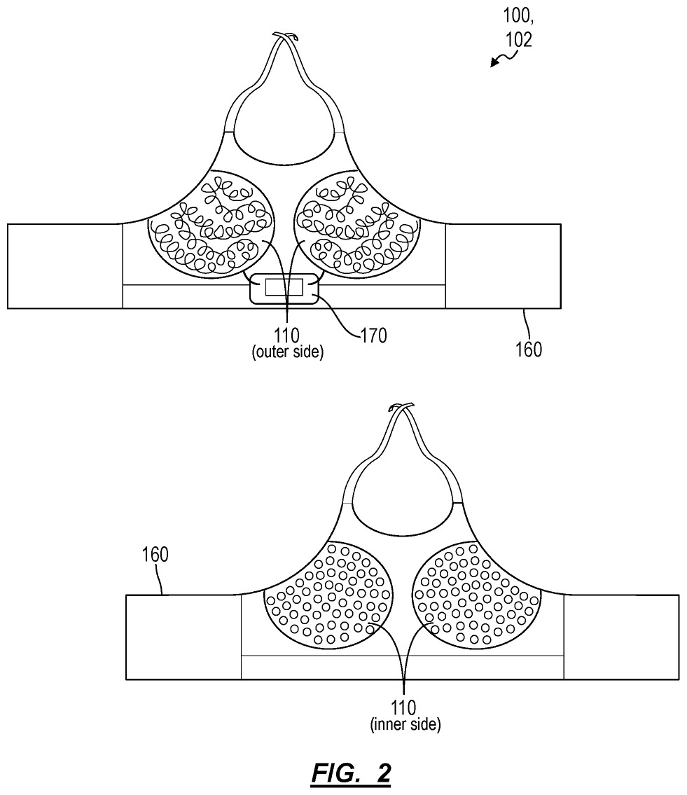

As shown in FIG. 2, the method S100 utilizes a temperature-sensing brassiere 102 to record surface temperature data at the breasts of a user. Generally, the temperature-sensing brassiere 102 includes a set of temperature sensors 134 that are evenly across surfaces of a user's breasts in a manner that facilitates normal circulation (e.g., similar to circulation in the user's breasts when the user is not wearing the temperature-sensing brassiere 102), thereby enabling collection of spatially consistent temperature data across multiple users characterized by variable breast sizes. In particular, the temperature-sensing brassiere 102 can include: a pair of sensor meshes 110 (one for each breast of the user) for distributing a set of temperature sensors 134 across each breast of the user; and a brassiere structure that locates each sensor mesh 110 over each breast of the user. The temperature-sensing brassiere 102 can further include: one or more IMUs arranged within the brassiere structure in order to detect changes in the user's posture during the data collection period and/or to detect the size and/or shape of the user's breasts; a controller 150, arranged within the brassier structure in order to sample data from the set of temperature sensors 134 in the temperature-sensing brassiere 102, and any IMUs of the temperature-sensing brassiere 102; and a battery arranged within the brassiere structure in order to provide electrical power to the controller 150.

More specifically, the temperature-sensing brassiere 102 can include a pair of sensor meshes 110 defining a mesh pattern (e.g., a triangle mesh, a quadrilateral mesh, a hybrid mesh) and each including a flexible mesh 120 structure (i.e. a flexible mesh 120) and a set of temperature-sensing assemblies 130. Thus, the temperature-sensing brassiere 102 can include: a first flexible mesh 120 extending from immediately right of a sternum of the user across a right breast of the user to a right axillary region of the user and from below the right breast of the user across the right breast of the user to a right clavicular region of the user, when the temperature-sensing brassiere 102 is worn by the user; and a second flexible mesh 120 extending from immediately left of a sternum of the user across a left breast of the user to a left axillary region of the user and from below the left breast of the user across the left breast of the user to a left clavicular region of the user, when the temperature-sensing brassiere 102 is worn by the user. Each of the first sensor mesh 110 and the second sensor mesh 110 can further include a set of temperature sensing assemblies arranged at intersections in the sensor meshes 110 (i.e. a first set of temperature-sensing assemblies 130 arranged at intersections in the first flexible mesh 120 and a second set of temperature-sensing assemblies 130 arranged at intersections in the second flexible mesh 120).

Additionally, the temperature-sensing brassiere 102 includes an electrical connection (i.e. an electrical trace 140 or wire) for each of the temperature-sensing assemblies 130 that electrically couples each temperature-sensing assembly 130 to the controller 150 via the flexible mesh 120, thereby enabling the controller 150 to sample a temperature sensor 134 in each temperature-sensing assembly 130 during the data collection period.

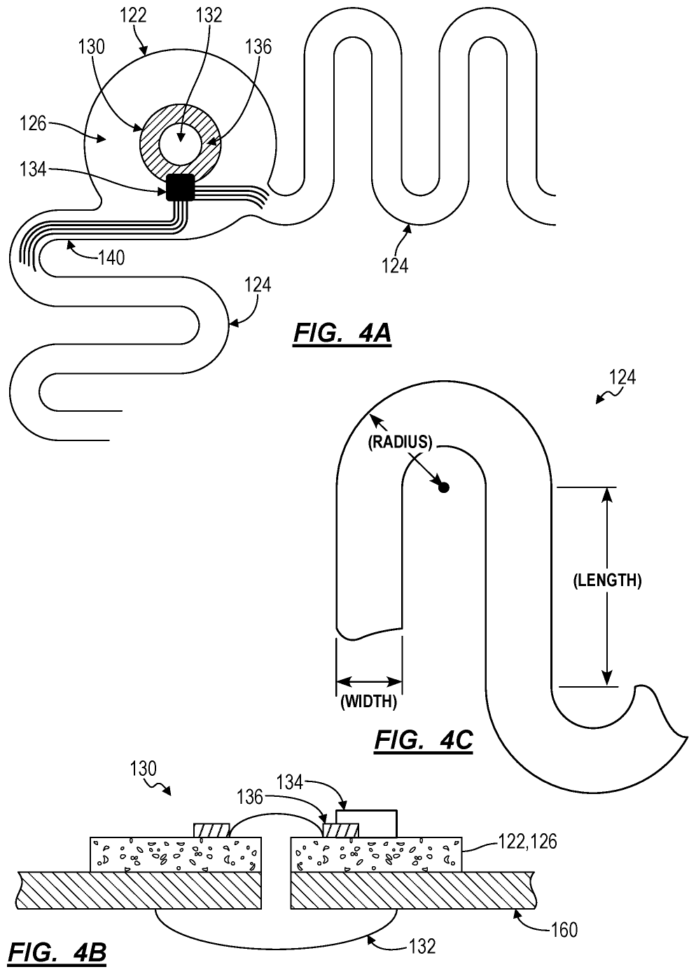

As shown in FIGS. 4A, 4B, and 4C, in one variation of the temperature-sensing brassiere 102, the flexible mesh 120 of the sensor mesh 110 includes a substrate 122 (such as a flexible PCB) configured in the shape of the mesh pattern. In this variation, each segment of the substrate 122 in the mesh pattern includes a boustrophedonic flexure (i.e. a boustrophedonic segment 124) configured to change in shape to accommodate a range of breast sizes. Furthermore, in this variation, the substrate 122 includes a set of island regions 126 at each intersection of the set of boustrophedonic segment 124 where the temperature-sensing assembly 130 mounts to the substrate 122. Each boustrophedonic segment 124 can also include an electrical trace 140 in the substrate 122 electrically coupling each of the temperature-sensing assemblies 130 to the controller 150.

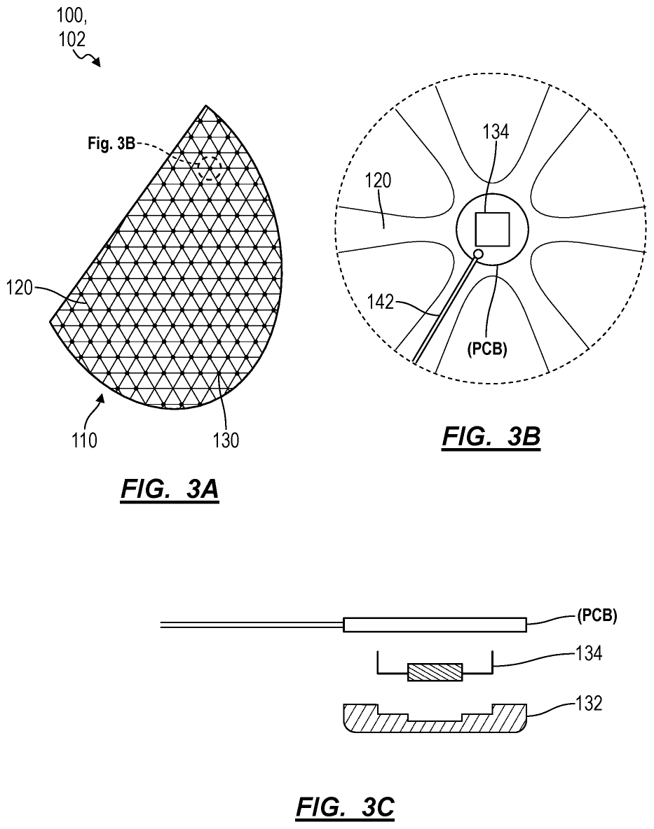

As shown in FIGS. 3A, 3B, and 3C, in another variation of the temperature-sensing brassiere 102, the flexible mesh 120 is a fabric mesh supporting temperature-sensing assemblies 130 at each intersection of the fabric mesh. This variation additionally includes elastic wires 142 arranged along the fabric mesh electrically coupling each temperature-sensing assembly 130 to the controller 150.

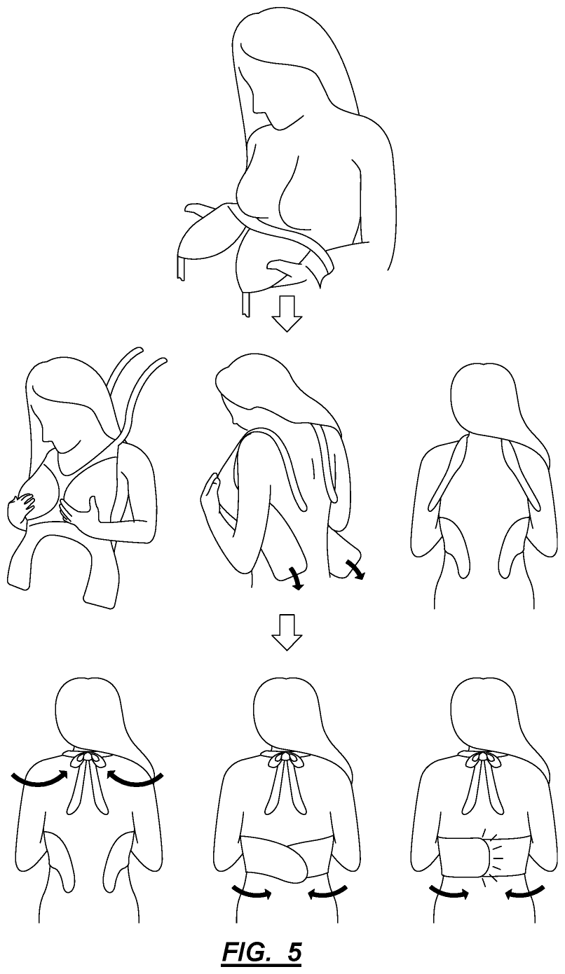

The temperature-sensing brassiere 102 can be secured to the user, as shown in FIG. 5, in a similar manner to a typical brassier. However, instead of a typical cup, the temperature-sensing brassiere 102 includes a pair of sensor meshes 110 for positioning a set of temperature sensors 134 across each of the user's breasts (i.e. a left mesh and a right mesh, or a single mesh for a post-mastectomy user). The temperature-sensing brassiere 102 can also include a power source, a controller 150, wireless chip (e.g. a Wifi or Bluetooth chip), and/or a wired port as means for recording, formatting, and/or transmitting data received from the temperature sensors 134 in the left sensor mesh 110 and the right sensor mesh 110 to another computational device 106 to perform post-processing and analysis of the temperature data.

### 4.1 Fabric Mesh Variation

As shown in FIG. 2, in one variation, the temperature-sensing brassiere 102 includes a left sensor mesh 110 and a right sensor mesh 110 covering the left breast of the user and the right breast of the user respectively. The left sensor mesh 110 includes an elastic material webbing that covers the left breast of the user. Horizontally, the left sensor mesh 110 attaches to the bridge of the temperature-sensing brassiere 102 proximal to the left side of the user's sternum and extends over the user's left breast to the left axillary region of the user (i.e. the underarm region of the user level with the breasts of the user), when the temperature-sensing brassiere 102 is worn by the user. Vertically, the left sensor mesh 110 attaches to the torso band of the temperature-sensing brassiere 102 and extends from under the user's left breast over the left breast of the user to the left clavicular region of the user. The right sensor mesh 110 of the temperature-sensing brassiere 102 is a mirrored version of the left sensor mesh 110 and extends over the corresponding regions on the right side of the user's body. The shapes defined by the sensor meshes 110 can approximate the anatomical shape of a typical breast. In one implementation, the sensor mesh 110 covers the breast with a teardrop-shaped mesh area that approximates the shape of a typical distribution of breast tissue on a female torso. However, the shape of the sensor mesh 110 area can be any shape that covers the user's breasts.

Within the sensor mesh 110 area defined by the temperature-sensing brassiere 102, the sensor mesh 110 itself includes an elastic fabric mesh or a webbing of elastic material, such as a fabric including hypoallergenic latex or an elastic polymer. The temperature-sensing assemblies 130 are arranged at intersections of the elastic webbing material, such that, when the temperature-sensing brassiere 102 is worn by a user, the webbing stretches to accommodate the user's breasts and distributes the temperature sensors 134 of the temperature-sensing assemblies 130 across the surface of the breast. In one implementation, the sensor mesh 110 defines a triangular mesh pattern, as shown in FIG. 3A. In alternative implementations, the sensor mesh 110 defines other mesh patterns such as a grid mesh, diamond mesh, or hexagonal mesh (i.e. honeycomb pattern). In yet another alternative implementation, the sensor mesh 110 defines a variable mesh, which has a variable pattern, such that the shape and size of the mesh pattern matches a curvature of a typical breast. Additionally, the variable mesh can define an increasing density of intersections in the mesh within subareas of the sensor mesh 110 area that correspond to anatomic regions with a higher incidence of breast cancer. For example, the variable mesh can define a higher density of mesh intersections in an area of the sensor mesh 110 area that approximately corresponds to the upper-outer quadrant of the user's breast when the temperature-sensing brassiere 102 is worn by a user in order increase the resolution of temperature data in that area due to its higher incidence of breast cancer when compared to other regions of breast tissue.

However, the sensor mesh 110 area can define any type of mesh structure or can define another flexible structure coupled to the temperature sensors 134.

### 4.2 Flexible Substrate Variation

As shown in FIG. 4A, in one variation, the temperature-sensing brassiere 102 includes a flexible substrate 122 (i.e. a flexible PCB), consisting of polyimide, polyether ether ketone (hereinafter “PEEK”), as the sensor mesh 110 of the temperature-sensing brassiere 102. In this variation, the substrate 122 is characterized as a one-piece (e.g., for each breast of the user) PCB construction in a mesh pattern, such as a triangular mesh, a grid mesh, a diamond mesh, or a hexagonal mesh. At each intersection of the mesh pattern, the substrate 122 can define island regions 126 on which each temperature-sensing assembly 130 is arranged on the substrate 122. Additionally, in order to increase the flexibility of the substrate 122, the substrate 122 can include boustrophedonic flexures as segments in the flexible mesh 120.

Thus, in this variation, each sensor mesh 110 in the temperature-sensing brassiere 102 includes: a substrate 122 that defines the mesh pattern and includes: a set of boustrophedonic segments 124 arranged in the mesh pattern; and a set of island regions 126 at each intersection of the set of boustrophedonic segments 124 in the mesh pattern. Additionally, in implementations including a pair of sensor meshes 110 (one for each breast of the patient), the temperature-sensing brassiere 102 can additionally include a second substrate 122 that defines the mesh pattern, which further includes: a second set of boustrophedonic segments 124 arranged in the mesh pattern; and a second set of island regions 126 at each intersection of the second set of boustrophedonic segments 124 in the mesh pattern. Alternatively, the temperature-sensing brassiere 102 can include a single substrate 122 that includes both sensor meshes 110 and therefore stretches across the sternum of the user (via a bridge in the brassiere structure) or around the back of the user via the brassiere structure to connect both sensor meshes 110 of the temperature-sensing brassiere 102.

### 4.2.1 Boustrophedonic Segment

As shown in FIGS. 4A and 4C, the substrate 122 can include a set of boustrophedonic segments 124 wherein each boustrophedonic segment 124 defines a boustrophedonic pattern and is configured to extend transverse to the boustrophedonic pattern in response to tension along the boustrophedonic segment 124 and wherein the substrate 122 is configured to expand over a breast of the user and conform to the breast of the user based on extension of the set of boustrophedonic segments 124. More specifically, each boustrophedonic segment 124 defines parallel (or near parallel) straight segments connected by curved segments on alternating ends of the parallel segments, thereby enabling the segment to extend and contract elastically in a direction transverse to the parallel segments as a flexure between island regions 126 of the substrate 122.

The boustrophedonic segment 124 can define a width, a length (of the parallel segments), a radius of curvature of the curved segments, and a number of parallel segments in each boustrophedonic segment 124. In one implementation, the boustrophedonic segment 124 can define different widths, lengths, curvatures, and/or numbers of parallel segments depending on the location of the boustrophedonic segment 124 in the sensor mesh 110 in order to confer tuned elastic properties at particular regions within the sensor mesh 110.

In one implementation, the substrate 122 can include segments defining other form factors configured to act as a flexure between island regions 126 in the sensor mesh 110 (e.g., a sinusoidal pattern, a sawtooth pattern).

The set of boustrophedonic segments 124 can include electrical traces 140 in order to electrically couple temperature-sensing assemblies 130 arranged on the island regions 126 of the substrate 122 to the controller 150 of the temperature-sensing brassiere 102. These electrical traces 140 are further described below.

### 4.2.2 Island Regions

The substrate 122 also includes island regions 126 at the intersection of the boustrophedonic segment 124 in order to support the temperature-sensing assemblies 130 in the sensor mesh 110. Generally, the island regions 126 can be circular in shape and are characterized by a surface area sufficient for a temperature sensor 134 (e.g., an integrated circuit) to be arranged on the substrate 122 within the island region 126. Each island region 126 can also include a center bore to enable thermal conductivity between the skin of the user and the temperature sensor 134 arranged on the surface of the substrate 122 via a thermal contact 132 positioned within the center bore of each island region 126.

In one implementation, the island region 126 includes a filleted interface between each boustrophedonic segment 124 and the island region 126 to reduce stress concentrations between each boustrophedonic segment 124 and the island region 126.

However, the set of island regions 126 of the substrate 122 can define any other shape that accommodates a temperature-sensing assembly 130 and can withstand tensile forces between the boustrophedonic segment 124 and the island regions 126.

### 4.2.3 Electrical Traces

As shown in FIG. 4A, the substrate 122 can include a set of electrical traces 140 electrically coupling each temperature-sensing assembly 130 to the controller 150 of the temperature-sensing brassiere 102. Generally, each electrical trace 140 traverses a set of boustrophedonic segments 124 in the substrate 122 thereby electrically coupling each of the temperature-sensing assemblies 130 to each other and then to the controller 150. More specifically, each electrical trace 140 can include a set of electrical lines including a positive voltage supply line; a negative voltage supply line; a serial data line; and a serial clock line. However, each electrical trace 140 can include additional electrical lines to support additional sensors or greater functionality of the temperature sensors 134. Additionally, each electrical trace 140 can mutually electrically couple each temperature-sensing assembly 130 to the same set of electrical lines in order to sample the sensor mesh 110 of the temperature-sensing brassiere 102 according to an I2C protocol or any other synchronous communication protocol for integrated circuits. Thus, the controller 150 can individually sample each temperature-sensing assembly 130 for a temperature value via the set of electrically traces traversing the boustrophedonic segment 124 of the temperature-sensing brassiere 102.

In one implementation, each temperature-sensing assembly 130 is electrically coupled to the controller 150 via a set of redundant electrical traces 140, each electrical trace 140 in the set of redundant electrical traces 140 traversing a unique subset of boustrophedonic segments 124 in the first set of boustrophedonic segments 124 to electrically couple the temperature sensor 134 to the controller 150. In order to electrically couple to the controller 150, each temperature-sensing assembly 130 includes a minimum of one electrical trace 140 traversing a single boustrophedonic segment 124 intersecting the island region 126, at which the temperature-sensing assembly 130 is located. However, in a mesh pattern, each island region 126 is coupled to multiple boustrophedonic segments 124 (e.g., six boustrophedonic segments 124 in a triangular mesh pattern). Therefore, each temperature-sensing assembly 130 in the sensor mesh 110 of a temperature-sensing brassiere 102 can connect with multiple electrical traces 140 (e.g., up to six electrical traces 140 in a triangular mesh pattern) traversing boustrophedonic segments 124 adjacent to the temperature-sensing assembly 130. Thus, if a boustrophedonic segment 124 is ruptured (or otherwise compromised) during use of the temperature-sensing brassiere 102, a temperature sensing assembly can maintain an electrical connection with the controller 150 via electrical traces 140 on adjacent boustrophedonic segments 124. A temperature-sensing brassiere 102 including redundant electrical traces 140 can therefore exhibit improved durability when utilized in a clinical setting or other application resulting in a high number of wear cycles.

Additionally, each sensor mesh 110 of the temperature-sensing brassiere 102 can include selectively routed electrical traces 140 across various boustrophedonic segments 124 in the sensor mesh 110. For example, a sensor mesh 110 can include electrical traces 140 on only a subset of boustrophedonic segments 124 adjacent to a temperature-sensing assembly 130 in order to electrically isolate various regions of the sensor mesh 110 from each other. Sensor meshes 110 including isolated regions of temperature sensing assemblies can exhibit improved serviceability upon malfunction of the temperature-sensing brassiere 102 by enabling a technician to more easily locate a malfunctioning temperature-sensing assembly 130 within an isolated region of the sensor mesh 110.

### 4.3 Sensing Assemblies

Generally, the temperature-sensing brassiere 102 includes a temperature-sensing assembly 130 at each intersection of each flexible mesh 120 of the temperature-sensing brassiere 102. Each temperature-sensing assembly 130 can include: a thermal contact 132 configured to contact the skin of a user; and a temperature sensor 134 configured to detect a temperature of the thermal contact 132. Depending on the implementation of the flexible mesh 120 and/or the brassiere structure of the temperature-sensing brassiere 102, the temperature-sensing brassiere 102 can include a set of temperature-sensing assemblies 130 that are configured to attach at intersections of the flexible mesh 120 and electrically couple with the controller 150.

More specifically, as shown in FIGS. 3B and 3C in variations of the temperature-sensing brassiere 102 including an elastic fabric mesh, the temperature-sensing assembly 130 can include a PCB coupled to the elastic fabric mesh (on the side of the elastic fabric mesh facing the skin of the user); a temperature sensor 134 mounted to the PCB (on the side of the PCB facing the skin of the user); and a thermally conductive cover enclosing the temperature sensor 134 and the PCB. The PCB of the temperature-sensing assembly 130 can then then be connected to elastic wires 142 that are imbedded in the elastic fabric mesh via ports in the thermally conductive cover in order to electrically couple the temperature-sensing assembly 130 to the controller 150 of the temperature-sensing brassiere 102.

As shown in FIG. 4B, in variations of the temperature-sensing brassiere 102 including a flexible substrate 122, the temperature-sensing brassiere 102 includes a set of temperature-sensing assemblies 130 arranged at island regions 126 of the substrate 122. Generally, in this variation, each temperature sensing assembly in the set of temperature sensing assemblies includes a thermal contact 132 fastened to the substrate 122 at the island region 126 via a bore in the substrate 122 at the island region 126; and a temperature sensor 134 coupled to the substrate 122 at the island region 126 and configured to detect the temperature of the thermal contact 132. Additionally, each temperature-sensing assembly 130 can include: a thermally conductive surface 136 coupled to the substrate 122 at the island region 126 concentric to the bore in the island region 126, contacting the thermal contact 132 of the temperature-sensing assembly 130, and contacting the temperature sensor 134. Thus, each temperature-sensing assembly 130 in the sensor mesh 110 functions to locate the temperature sensor 134 within the sensor mesh 110 of the temperature-sensing brassiere 102 and contacting a thermal contact 132 such that the temperature sensor 134 can detect the temperature of the thermal contact 132 and therefore estimate the temperature of the skin of the user at the location of the thermal contact 132.

In one implementation, a subset of temperature sensing assemblies in the set of temperature-sensing assemblies 130 can include IMUs (as integrated circuits coupled to the flexible substrate 122). In this implementation, the temperature-sensing brassiere 102 can include additional electrical traces 140 and/or other circuit elements according to specifications of the IMU.

The temperature-sensing assembly 130 can further include any other circuit elements—such as resistors (e.g., pull-up resistors or pull-down resistors), transistors, switches—according to specifications of the temperature sensors 134 or other electrical components included in the temperature-sensing assembly 130.

### 4.3.1 Temperature Sensors

As shown in FIGS. 3B, 3C, and 4B the temperature-sensing brassiere 102 includes temperature sensors 134 arranged at intersections in the sensor mesh 110. The temperature sensors 134 are digital medical-grade contact temperature sensors 134. In some implementations, the temperature sensors 134 have a temperature resolution of at least 0.1° C. The temperature sensors 134 are electrically coupled to the controller 150, which is also included in the temperature-sensing brassiere 102. In variations of the temperature-sensing brassiere 102 including an elastic fabric mesh, the temperature sensors 134 are connected to the controller 150 via elastic wiring throughout the sensor mesh 110. Although the resistance of the elastic wires 142 may change as the elastic wires 142 stretch along with the sensor mesh 110, the digital temperature signal remains readable at the controller 150 and the values read at the controller 150 remain accurate.

In one implementation, each temperature sensor 134 is transiently coupled to the interior of the sensor mesh 110 via any suitable attachment method, such that the temperature sensors 134 can be removed from the sensor mesh 110 to enable cleaning of the temperature-sensing brassiere 102 without exposing the electrical components to water or cleaning agents.

In variations of the temperature-sensing brassiere 102 including the substrate 122 mesh, the temperature sensors 134 are electrically coupled to the controller 150 via electrical traces 140 on the substrate 122.

In one implementation, the temperature sensors 134 are not placed on every intersection of the sensor mesh 110 and are instead concentrated in regions of the sensor mesh 110 that coincide with regions of the breast that have a higher incidence of breast cancer (e.g. the outer-upper quadrant). In another implementation, the temperature-sensing brassiere 102 includes an arrangement of temperature sensors 134 based on a sensitivity analysis of the machine learning model. In this implementation, the machine learning model is reevaluated without data from each of the temperature sensors 134 in the sensor mesh 110. Temperature sensors 134 that have little effect on the sensitivity of the machine learning model can then be removed from the sensor mesh 110. This process results in a more efficient arrangement of temperatures sensors 134.

In one implementation, the temperature-sensing brassiere 102 includes external labels for each temperature sensor 134 arranged on the sensor mesh 110. The external label can be an outward facing number individually identifying each temperature sensor 134 in the left or right sensor meshes 110. The external labels can identify corresponding sensors between the left sensor mesh 110 and the right sensor mesh 110. For example, a temperature sensor 134 on the left sensor mesh 110 can have the same label as a temperature sensor 134 on the right sensor mesh 110 to indicate that the two sensors are at the same relative location in the left and right mesh. The external labels for each temperature sensor 134 facilitate identification of the temperature sensors 134 that are nearest to the user's nipples and facilitate consistent alignment of the user's breasts in the left and right sensor meshes 110 prior to data collection from the temperature-sensing brassiere 102.

In one implementation, the temperature-sensing brassiere 102 includes 192 temperature sensors 134 across the left sensor mesh 110 and the right sensor mesh 110 (96 temperature sensors 134 in each sensor mesh 110), thereby providing high-resolution temperature data to the computational device 106 for further processing.

In variations of the temperature-sensing brassiere 102 including the substrate 122 mesh, the temperature sensors 134 are electrically coupled to the controller 150 via electrical traces 140 on the substrate 122 and are directly attached to the substrate 122, thereby reducing manufacturing costs. In one implementation, the temperature sensors 134 are attached to the substrate 122 opposite the skin of the user and are configured to detect the temperature of the skin of the user via a thermally conductive thermal contact 132 further described below.

However, the temperature sensors 134 can be of any type, can be characterized by any other sensitivity or dynamic range, and can be attached to the sensor mesh 110 or bra cup in any other way.

### 4.3.2 Thermal Contact

As shown in FIGS. 3C and 4B, in some implementations, each temperature-sensing assembly 130 in the set of temperatures-sensing assemblies includes a thermal contact 132 fastened to the substrate 122 at the island region 126 via a bore in the first substrate 122 at the island region 126. Therefore, the thermal contact 132 extends from an externally facing (i.e. away from the skin of the user) surface of the substrate 122 to an internally facing side of the substrate 122 in order to: approach thermal equilibrium with the user's skin; and enable the temperature sensor 134 to measure the temperature at the user's skin without directly touching the user's skin. The thermal contact 132 can be manufactured from aluminum or an aluminum alloy, or any other material with a high thermal conductivity such as copper, silver, copper alloy, silver alloy.

In one implementation, the thermal contact 132 defines two regions: a rounded or smooth head with an internal face configured to contact the skin of the user; and a mandrel extending from the head and through the bore in the substrate 122. The mandrel of the thermal contact 132 can function as a rivet and therefore can be fastened to the substrate 122 via riveting opposite the head of the thermal contact 132. However, the thermal contact 132 can be fastened to the substrate 122 in any other way.

### 4.3.3 Thermally Conductive Surface

As shown in FIG. 4A, each temperature-sensing assembly 130 in the set of temperature-sensing assemblies 130 can include a thermally conductive surface 136: coupled to the first substrate 122 at the island region 126 concentric to the bore in the island region 126; contacting the thermal contact 132 of the temperature-sensing assembly 130; and contacting the temperature sensor 134 of the sensing assembly. Thus, the thermally conductive surface 136 enables thermal conductivity between the thermal contact 132 and the temperature sensor 134 such that the temperature sensor 134 can accurately measure the temperature of the thermal contact 132.

Depending on the implementation and/or the particular arrangement of the temperature sensor 134 around the bore in the island region 126 of the substrate 122, the thermally conductive surface 136 can define various shapes on the surface of the substrate 122 in order to thermally couple the thermal contact 132 and the temperature sensor 134 in the temperature-sensing assembly 130.

### 4.4 Brassiere Structure and Material

Generally, the straps, bands, and attachment mechanisms of the temperature-sensing brassiere 102 can be implemented in any arrangement that effectively places the sensor mesh 110 over the breasts of the user.

In one implementation, the temperature-sensing brassiere 102 includes left and right sensor meshes 110, a bridge connecting the left and right sensor meshes 110 across the sternum of the user, an adjustable elastic torso band extending from the left and right sensor meshes 110 that clasp or attach (e.g. via a hook and loop attachment mechanism) behind the back of the user, and adjustable elastic shoulder straps that clasp or tie behind the neck of the user. In this implementation, the temperature-sensing brassiere 102 is highly adjustable while also applying consistent contact force between each temperature sensor 134 in the sensor mesh 110 and the surface of the user's breasts.

In another implementation, the brassiere structure defines an adjustable front wrap style closure and is manufactured from an elastic fabric characterized by lower elasticity in order to maintain a similar level of pressure on the breast of each user of the temperature-sensing brassiere 102 independent of the breast size of each user. The brassiere structure defining an adjustable front wrap style closure is characterized by a continuous strap along the back of the user connected to a left wing and a right wing of the brassiere structure. The right wing of the brassiere structure supports the right sensor mesh 110 and defines an attachment end that engages with any of a set of attachment points on the interior of the left wing (e.g., near the underarm region of the user) and, therefore, wraps around the right breast of the user and under the left breast of the user to attach under the left arm of the user. The left wing of the brassiere structure supports the left sensor mesh 110 and defines an attachment end that wraps over the right wing around the front of the user and engages with a set of attachment points on the exterior of the right wing of the brassiere structure under the right arm of the user. Alternatively, the above description can apply vice versa, and therefore the brassiere structure can include a right wing that wraps over the left wing.

However, the configuration of the brassiere's straps, bands, attachment points, etc. can include any brassiere design that provides consistent contact between the temperature sensors 134 in the sensor mesh 110 and the breasts of the user.

Each component of the temperature-sensing brassiere 102 can consist of a different fabric or fabric blend specific to the function of that component. Generally, the temperature-sensing brassiere 102 consists of elastic fabrics that conform to the shape of the user's torso, back, and breasts. For example, the temperature-sensing brassiere 102 can consist of various spandex or elastane blends, rubber or latex fabrics, or neoprene rubber fabrics. Alternatively, the temperature-sensing brassiere 102 can include various weaves of non-elastic fabrics.

In one implementation, the temperature-sensing brassiere 102 can include thermal insulation over the sensor meshes 110. The thermal insulation can increase the rate of blood flow into the user's breasts when the temperature-sensing brassiere 102 is secured to the user, which may be beneficial in detecting temperature anomalies in the user's breasts.

In one implementation, the temperature-sensing brassiere 102 is a one-size-fits-all design consisting of an elastic fabric. In another implementation, the brassiere is adjustable to provide consistent contact pressure across the temperature sensors 134 in the sensor meshes 110.

However, the temperature-sensing brassiere 102 can consist of any fabric or mix of fabrics that enables adjustability and consistent pressure between the temperature sensors 134 in the sensor mesh 110 and the surface of the user's breasts.

In variations of the temperature-sensing brassiere 102 including a substrate 122, the substrate 122 can attach to the brassiere structure on the exterior of the brassiere structure such that the substrate 122 is coupled to the brassiere structure by each of the thermal contacts 132 passing through the brassiere structure and successively a corresponding bore in each of the set of island regions 126 of the substrate 122. Thus, the temperature-sensing brassiere 102 can include a brassiere structure interposed between the substrate 122 and the thermal contact 132 of each temperature-sensing assembly 130 in the set of temperature-sensing assemblies 130, wherein the thermal contact 132 of each temperature-sensing assembly 130 in the set of temperature-sensing assemblies 130 protrudes through the substrate 122 and the brassiere structure to contact the skin of the user proximal the breast region of the user.

Additionally, in one implementation, the system 100 can include a set of temperature-sensing brassiere 102 of various sizes (e.g., a small, medium, and large size) in order to fit a variety of users according to approximate cup size and chest size. In some implementations, the temperature-sensing brassiere 102 is modular in structure, such that various components, like the sensor mesh 110, bridge, torso band, and straps can be interchangeable with equivalent components of different sizes or shapes.

However, the temperature-sensing brassiere 102 can include a brassiere structure attached or integrated with the sensor mesh 110 according to any known attachment method that positions the sensor mesh 110 over the breasts of a variety of users when the temperature-sensing brassiere 102 is worn by those users.

### 4.5 IMU Configuration and Placement

In one variation of the temperature-sensing brassiere 102, the temperature-sensing brassiere 102 includes IMUs arranged on the sensor mesh 110. Generally, the IMUs function to provide data to the processor for estimating size, shape, and mass of a user's breasts when combined with an anatomical model. The IMUs can also indicate the posture of the user during data collection, which can affect the temperature of the user's breasts and can be controlled during data collection.

The IMUs, like the temperature sensors 134, provide a digital output and can be electrically coupled to the processor via elastic wires 142 or deposited directly onto a flexible substrate 122 depending on the variation of the temperature-sensing brassiere 102.

In one implementation, the temperature-sensing brassiere 102 includes IMUs placed proximate to the center of each quadrant of the sensor mesh 110, wherein each quadrant of the sensor mesh 110 roughly corresponds with the typical location of a quadrant of a user's breast when the temperature-sensing brassiere 102 is worn by a user. In an alternative implementation, the IMUs can be placed in horizontal alignment with the nipple of the user when the temperature-sensing brassiere 102 is worn by the user.

In one implementation, the temperature-sensing brassiere 102 includes an IMU placed on the back of the temperature-sensing brassiere 102 (e.g. on the torso band). This IMU provides specific data indicating the posture of the user during data collection. Therefore, in this implementation, the temperature-sensing brassiere 102 includes an inertial measurement unit: arranged in the brassiere structure; electrically coupled to the controller 150; and configured to measure a posture of the user during the data collection period.

However, IMUs can be placed in other configurations over the temperature-sensing brassiere 102 in order to estimate the size, shape, and/or mass of a user's breasts and/or the user's posture during data collection from the temperature-sensing brassiere 102.

### 4.6 Controller 150

In one implementation, the temperature-sensing brassiere 102 includes a controller 150 arranged within the brassiere structure. More specifically, the controller 150 can include a processor (or microprocessor) configured to: sample the temperature sensor 134 of each sensing assembly in the first set of sensing assemblies during a data collection period to generate a series of temperature data; and store the series of temperature data. Additionally, the controller 150 can include a multiplexer in order to sample various subsets of the temperature sensors 134 in the pair of sensor meshes 110.

In one implementation, the controller 150 is configured to implement the inter-integrated circuit protocol (hereinafter the “I2C protocol”), or any other synchronous digital communication protocol, in order to sequentially sample each temperature sensor 134 and IMU sensor included in the temperature-sensing brassiere 102.

Additionally, the controller 150 can include a common junction and wiring harness that can enable a wired connection with an external device such as a computer or specifically designed hub 104 such that the computer or hub 104 can: interface with the controller 150; extract temperature data from the controller 150; and/or reconfigure the controller 150.

In another implementation, the controller 150 includes a touchscreen for issuing commands to the temperature-sensing brassiere 102 or displaying that status of the temperature-sensing brassiere 102. Additionally or alternatively, the controller 150 can be electrically coupled to LED indicator lights, which may indicate additional or redundant status information. The controller 150 can also include buttons for calibrating the temperature-sensing brassiere 102, initiating the data collection period, turning the temperature-sensing brassiere 102 on or off, or any other function.

### 4.7 Remote Hub

In one implementation, the system 100 can include a hub 104 remote from the temperature-sensing brassiere 102 configured to: extract the series of temperature data from the temperature-sensing brassiere 102; upload the series of temperature data to a computational device 106; and erase the series of temperature data from the temperature-sensing brassiere 102. Additionally, the hub 104 can be configured to hold or otherwise support the temperature-sensing brassiere 102 when connected to the temperature-sensing brassiere 102 and extracting data from the temperature-sensing brassiere 102. Furthermore, the hub 104 can be configured with a wireless communication chip (e.g., BLUETOOTH or WIFI) and can communicate with computational devices 106 with access to the internet or directly with remote server 108 on the internet in order to upload the series of temperature data for analysis at a remote server 108 or another computational device 106. Alternatively, the hub 104 can connect to a computational device 106 via a wired connection.

### 4.8 Remote Data Processing

In one implementation, the system 100 can include a remote computational device 106 configured to: evaluate a breast cancer risk assessment model based on the series of temperature data; and estimate a breast cancer risk assessment for the user. In this implementation, the system 100 analyzes temperature data measured at the skin of the patient via the temperature-sensing brassiere 102 during a data collection period, the temperature-sensing brassiere 102 can then either directly transfer the series of temperature data to a remote computational device 106 or can transfer the series of temperature data to an intermediate device such as the aforementioned hub 104 or another intermediate device. Upon receiving the series of temperature data from the temperature-sensing brassiere 102, the remote computational device 106 can execute a breast cancer risk assessment model, further described below, to calculate a breast cancer risk assessment for the user of the temperature-sensing brassiere 102.

### 5. Test Preparation

Before the system 100 executes the method S100, the user and temperature-sensing brassiere 102 are prepared for the test. The user may be instructed to sit in a temperature-controlled environment (e.g. an examination room) for an acclimatization period. Additionally, the user may be instructed to disrobe from the waist up in order to expose the user's breasts to the temperature-controlled environment.

Additionally, the exposure of the user's breasts to a relatively cold environment may initiate a “cold shock” effect, wherein capillaries in the user's breasts may restrict blood flow to the surface of the skin. This cold shock effect may be beneficial in detecting temperature anomalies as blood flow returns to the surface of the user's breasts because differences in the warming rates of similar regions between the two breasts can indicate an anomaly. Thus, the duration of the acclimatization period can be sufficiently long to allow the cold shock effect to restrict blood flow in the user's capillaries.

While the user is acclimating to the temperature-controlled environment, the method S100 can include calibrating the temperature-sensing brassiere 102. The calibration process can include leaving the temperature-sensing brassiere 102 with the temperature sensors 134 contacting a sanitized surface in the temperature-controlled environment for a particular calibration period. Alternatively, the temperature sensors 134 can be exposed to the air in the temperature-controlled environment. Upon receiving a command from a computational device 106 connected to the temperature-sensing brassiere 102 or via a direct interaction with the temperature-sensing brassiere 102 (e.g. a button press), the temperature-sensing brassiere 102 can calibrate the temperature sensors 134 and, optionally, any IMUs arranged on the temperature-sensing brassiere 102.

In implementations wherein the temperature-sensing brassiere 102 includes IMUs, the IMUs can calibrate based on the force of gravity, such that when the temperature-sensing brassiere 102 is secured to the user, the IMUs can provide accurate positional data.

However, the user and the temperature-sensing brassiere 102 can be prepared for the method S100 in any other way.

### 6. Data Collection

The temperature-sensing brassiere 102 can begin collecting data by initiating a data collection period approximately (e.g. immediately before or after) when the temperature-sensing brassiere 102 is secured to the user. Alternatively, the temperature-sensing brassiere 102 can initiate the data collection period upon being disconnected from the hub 104 or in response to receiving a button press or touchscreen interaction on the temperature-sensing brassiere 102. While securing the temperature-sensing brassiere 102 to the user, care may be taken to adjust the temperature-sensing brassiere 102 such that the temperature sensors 134 (or corresponding thermal contacts 132) in the sensor meshes are properly distributed across the surface of the user's breasts. Additionally or alternatively, the computational device 106 connected to the temperature-sensing brassiere 102 can receive an input from a medical professional administering the test indicating the locations or identifying information of the sensor closest to the user's left nipple and the sensor closest to the user's right nipple.

Before initiating the data collection period, the user may be instructed by the medical professional administering the test, or via a user interface of the computational device 106 connected to the temperature-sensing brassiere 102, to maintain a particular posture during the data collection period. In one implementation, the method S100 includes displaying a notification on the computational device 106 connected to the temperature-sensing brassiere 102 that a user is not maintaining the correct posture as determined by IMUs in the temperature-sensing brassiere 102. In another implementation, the method S100 includes storing temperature data from the temperature-sensing brassiere 102 in association with IMU data indicating the posture of the user during the data collection period.

Upon initiating the data collection period, the method S100 includes collecting data from the temperature sensors 134 in the set of temperature-sensing assemblies 130 in the left and right sensor meshes 110 in the temperature-sensing brassiere 102. In one implementation, the temperature sensors 134 collect data at 16 Hz. In one implementation, the method S100 includes storing the temperature data locally (e.g. on an SD card) at the temperature-sensing brassiere 102 before transmitting the temperature data to the computational device 106, either wirelessly or via a wired connection. In one implementation, the method S100 includes storing gyroscopic, acceleration, and or positional data from IMUs arranged on the temperature-sensing brassiere 102.

Thus, the system 100 can, during a data collection period, record breast temperature data from each temperature-sensing assembly 130 in the first set of temperature-sensing assemblies 130 and second breast temperature data from the second set of temperature-sensing assemblies 130.

In one implementation, the temperature data is stored in a matrix format, such that each cell in the matrix indicates temperature data from a particular temperature sensor 134 in the sensor mesh 110, which is also associated with a known relative position in the sensor mesh 110 of the particular temperature sensor 134. Additionally, the temperature data can include a series of matrices for each sampling period of the temperature sensors 134 in the sensor meshes 110. In an alternative implementation, the temperature data can be stored as a set of arrays, in which each array corresponds to a set of temperature data (or IMU data) from the sensor meshes 110 during a sampling period.

The duration of the data collection period can vary depending on the implementation of the method S100 (e.g. 5 minutes, 15 minutes, 60 minutes, etc.). Upon completion of the data collection period, the temperature-sensing brassiere 102 can be removed from the user.

However, the method S100 can include any means of extracting temperature data from the temperature-sensing brassiere 102.

### 6.1 I2C Sampling

In Block S110, the controller 150 can execute an I2C protocol in order to sample each temperature sensor 134 in each temperature-sensing assembly 130. By executing an I2C protocol, the controller 150 allows for simple circuit design (e.g., by including a positive voltage supply, a negative voltage supply, a serial data channel, and a serial clock channel). Each temperature sensor 134 in the pair of sensor meshes 110 of the temperature-sensing brassiere 102 can be assigned with an individual bit address according to the I2C protocol.

However, in implementations where the temperature-sensing brassiere 102 does not include temperature sensors 134 with a bit address long enough to cover the total number of sensors between the two sensor meshes 110, the controller 150 can utilize a multiplexer to switch between separate subsets of temperature sensors 134. In this implementation, the temperature-sensing brassiere 102 can include separate serial data channels and/or serial clock channels for each subset of the temperature sensors 134. Thus, the temperature-sensing brassiere 102 can include a multiplexer such that each temperature sensor in the temperature-sensing brassiere 102 is individually addressable by the controller 150.

### 7. Data Processing and Representation

Once the data collection period is complete, various data processing steps can be executed at the controller 150 of the temperature-sensing brassiere 102, at the remote hub 104, at a computational device 106, and/or at a server connected to the computational device 106. Generally, the temperature data collected during the data collection period is spatially normalized according to the relative positions of the user's breasts in the sensor meshes 110; spatially interpolated based on an estimated size and shape of the user's breasts; displayed in a two or three-dimensional graphical representation; and/or analyzed to determine a breast cancer risk assessment for the user.

The temperature-sensing brassiere 102 collects a time series of temperature data from each temperature sensor 134 in the sensor meshes 110. As such, post data processing, the method S100 generates a dynamic two-dimensional or three-dimensional temperature profile of a user's breasts over the data collection period. Thus, any graphical representation of the data can be displayed in series or “played back” at real time or faster than real time in order to provide a dynamic visualization of temperature variations in the user's breasts over the data collection period.

In one implementation, the method S100 can include prompting the user to retake the breast cancer diagnostic test in response to detecting an incorrect posture of the user during the data collection period. More specifically, the method S100 can include: at a temperature-sensing brassiere 102 including an inertial measurement unit arranged in the brassiere structure; during the data collection period, recording a series of inertial data from the inertial measurement unit; and, after the data collection period, in response to estimating a noncompliant posture of the user based on the series of inertial data, prompting the user to reinitiate the breast cancer risk assessment.

### 7.1 Spatial Normalization

Generally, Block S120 of the method S100 includes spatially normalizing the temperature data from the temperature-sensing brassiere 102 during the data collection period. By spatially normalizing the data, the method S100 ensures that differences in position, size, or shape between the left and right breasts of the user have a minimal effect on the efficacy of the temperature data. For example, if a right nipple of the user was located at a first sensor in the right sensor mesh 110, then one would expect the left nipple of the user to be located at the corresponding sensor in the left sensor mesh 110. However, this is not always the case and the left and right breasts may be positioned quite differently in their respective sensor meshes 110. As such, spatial normalization shifts the temperature data in one sensor mesh 110 to more closely anatomically match the data from the other sensor mesh 110 (e.g. such that corresponding data streams represent temperature data from the same anatomical region on each breast).

The system 100 can: calculate the relative positional difference of a sensor in the left sensor mesh 110 closest to the left nipple of the user and a sensor in the right sensor mesh 110 closest to the right nipple of the user; and shift the temperature data from one sensor by the relative positional distance.

In an alternative implementation, a matrix transformation based on the anatomical properties of breasts is applied to one matrix based on the positional difference between the location of the user's nipples in each sensor mesh 110 in order to account for the anatomical curvature of the user's breast in the temperature-sensing brassiere. Therefore, the system 100 can shift the associated location of each cell in the matrix of temperature data by a different distance based on the relative position of the user's nipple in the sensor mesh. In this implementation, the matrix transformation can interpolate between data in the pre-transformed matrix to calculate values in the spatially normalized matrix.

In another alternative implementation, the temperature data from each set of temperature sensors 134 in each sensor mesh 110 is first interpolated before being spatially shifted. In this implementation, a function that takes in the interpolated temperature data from each sensor mesh 110 and the relative positional difference between the user's breasts at the nipple and outputs a new temperature profile based on the positional difference.

Thus, the method S100 can include spatially interpolating the first set of breast temperature data and the second set of breast temperature data to generate a first breast temperature profile and a second breast temperature profile.

However, the method S100 can include any other form of spatial normalization to adjust for differences in position between the left and right breasts relative to the sensor mesh 110. Additionally, any of the processes for spatial normalization can be applied individually to each sample of the temperature data over the data collection period.

### 7.2 Low-Resolution, Two-Dimensional Representation

In Block S140, once the temperature data has been spatially normalized, the method S100 includes displaying the temperature data as a time series on the computational device 106. In one implementation, the computational device 106 displays a set of colored dots, each dot in the set of colored dots representing a temperature sensor 134 in the left and right sensor meshes 110 of the temperature-sensing brassiere 102, wherein each dot is colored based on the temperature value from the corresponding sensor. A medical professional or other user can then view the sensor data from a computational device 106 connected to the temperature-sensing brassiere 102.

### 7.3 High-Resolution, Two-Dimensional Representation

In one implementation in Block S140, the method S100 includes displaying a two-dimensional high-resolution interpolation of the temperature data from each of the sensor meshes 110. In this implementation, the method S100 can include two-dimensional linear interpolation between the sensor data points. The two-dimensional representation can be displayed in roughly the shape of a breast or in the shape of the distribution of temperature sensors 134 in the sensor meshes 110. In one implementation, the linearly interpolated two-dimensional representation is displayed as a colored heat map on the computational device 106. Additionally or alternatively, the method S100 also includes displaying a difference profile illustrating temperature differences between the two sets of aligned temperature data.

### 7.4 Basic Three-Dimensional Representation

In one implementation, in Block S140, the method S100 includes displaying the interpolated temperature data from the temperature-sensing brassiere 102 in a basic three-dimensional representation. In this implementation, the interpolated temperature data is applied to a generic three-dimensional anatomical model of female breasts. The interpolated temperature data is centered on the model, such that the left and right nipples of the three-dimensional model display temperature data from the sensors closest to the user's left and right nipples respectively. The basic three-dimensional model provides some physical context to the temperature data and aids in analysis and diagnosis.

7.5 Three-Dimensional Representation with Custom Anatomical Model

In one implementation, in Block S140, the method S100 includes generating a custom anatomical model based on data from IMUs arranged on the temperature-sensing brassiere 102. In this implementation, the method S100 includes fitting a parametric anatomical model of a breast to the positional and gyroscopic data taken from the surface of each of the user's breasts. The parametric anatomical model produces a set of three-dimensional surfaces that represent a best fit for the positional and gyroscopic data. The method S100 then includes displaying the temperature data over the three-dimensional surface representing the user's breasts. As described above, the temperature data is centered at the nipple to ensure accurate representation of the temperature data.

### 7.6 Risk Assessment Via Machine Learning Model

In Block S130, the method S100 includes automatically calculating a breast cancer risk assessment for the user based on the temperature data based on a machine learning model. The machine learning model can include a neural network or any other statistical method for classification of the temperature data. In one implementation, the machine learning model outputs a binary score indicating further tests are necessary or that the user has a low risk of breast cancer. Alternatively, the machine learning model outputs a risk factor or risk categorization based on the input temperature data (e.g., such as a BI-RADS score). Additionally or alternatively, the machine learning model can display a two-dimensional or three-dimensional representation of the user's breasts and highlight the location of potentially cancerous tissue.

Thus, in Block S132, the method S100 can include: estimating a breast cancer risk assessment for the user based on the first breast temperature data and the second breast temperature data. further comprising estimating a location of an abnormal mass in the first breast of the user or the second breast of the user; and wherein rending the first breast temperature profile and the second breast temperature profile further comprises rendering an indication of the location of the abnormal mass in the first breast temperature profile or the second breast temperature profile in Block S142.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A system for evaluating breast cancer risk in a user comprising:
a temperature-sensing brassiere comprising:
a first substrate defining a mesh pattern and comprising:
a first set of boustrophedonic segments arranged in the mesh pattern; and
a first set of island regions at each intersection of the first set of boustrophedonic segments in the mesh pattern;
a first set of temperature-sensing assemblies, each temperature-sensing assembly in the first set of temperature-sensing assemblies arranged at an island region in the first set of island regions and comprising:
a thermal contact fastened to the first substrate at the island region via a bore in the first substrate at the island region; and
a temperature sensor coupled to the first substrate at the island region and configured to detect the temperature of the thermal contact;
a brassiere structure:
configured to locate the first substrate over a first breast region of the user; and
interposed between the first substrate and the thermal contact of each temperature-sensing assembly in the first set of temperature-sensing assemblies, wherein the thermal contact of each temperature-sensing assembly in the first set of temperature-sensing assemblies protrudes through the first substrate and the brassier structure to contact skin of the user proximal the first breast region of the user; and
a controller electrically coupled to the set of temperature sensors via the first substrate and configured to:
sample the temperature sensor of each temperature-sensing assembly in the first set of temperature-sensing assemblies during a data collection period to generate a series of temperature data; and
store the series of temperature data.

2. The system of claim 1 further comprising:
a hub configured to:
extract the series of temperature data from the temperature-sensing brassiere;
upload the series of temperature data to a computational device; and
erase the series of temperature data from the temperature-sensing brassiere; and
the computational device configured to:
evaluate a breast cancer risk assessment model based on the series of temperature data; and
estimate a breast cancer risk assessment for the user.

3. The system of claim 2, wherein the computational device is further configured to:
associate the series of temperature data with relative locations in the first breast region of the user;
spatially interpolate the series of temperature data to generate a breast temperature profile; and
render the breast temperature profile as a heat map representing a temperature of the first breast region of the user.

4. The system of claim 1, wherein the first substrate is characterized by one piece flexible printed-circuit-board construction.

5. The system of claim 1:
wherein the temperature-sensing brassiere further comprises:
a second substrate defining the mesh pattern and comprising:
a second set of boustrophedonic segments arranged in the mesh pattern; and
a second set of island regions at each intersection of the second set of boustrophedonic segments in the mesh pattern;
a second set of temperature-sensing assemblies, each temperature-sensing assembly in the second set of temperature-sensing assemblies arranged at an island region in the second set of island regions and comprising:
a thermal contact fastened to the second substrate at the island region via a bore in the second substrate at the island region; and
a temperature sensor coupled to the second substrate at the island region and configured to detect the temperature of the thermal contact;
wherein the brassiere structure is further configured to locate the second substrate over a second breast region of the user; and
wherein the brassiere structure is interposed between the second substrate and the thermal contact of each temperature-sensing assembly in the second set of temperature-sensing assemblies, wherein the thermal contact of each temperature-sensing assembly in the second set of temperature-sensing assemblies protrudes through the second substrate and the brassier structure to contact skin of the user proximal the second breast region of the user.

6. The system of claim 1, wherein the brassiere structure defines an adjustable front wrap closure style.

7. The system of claim 1:
wherein each boustrophedonic segment in the first set of boustrophedonic segments defines a boustrophedonic pattern and is configured to extend transverse to the boustophedonic pattern in response to tension along the boustrophedonic segment; and
wherein the first substrate is configured to expand over the breast of the user and conform to the breast of the user based on extension of the first set of boustrophedonic segments.

8. The system of claim 1, wherein each temperature-sensing assembly in the first set of temperature-sensing assemblies further comprises:
a thermally conductive surface:
coupled to the first substrate at the island region concentric to the bore in the island region;
contacting the thermal contact of the temperature-sensing assembly; and
contacting the temperature sensor of the temperature-sensing assembly.

9. The system of claim 1, wherein the first substrate defines a triangular mesh pattern.

10. The system of claim 1, wherein the controller is electrically coupled to each temperature sensor in the set of temperature sensors via an electrical trace traversing a subset of the boustrophedonic segments of the first substrate.

10. The system of claim 10:
wherein the electrical trace comprises:
a positive voltage supply;
a negative voltage supply;
a serial data line; and
a serial clock line; and
wherein a subset of the set of temperature sensors are mutually electrically coupled to the positive voltage supply, the negative voltage supply, the serial data line, and the serial clock line.

12. The system of claim 10, wherein a temperature sensor of a temperature-sensing assembly in the first set of temperature-sensing assemblies is electrically coupled to the controller via a set of redundant electrical traces, each electrical trace in the set of redundant electrical traces traversing a unique subset of boustrophedonic segments in the first set of boustrophedonic segments to electrically couple the temperature sensor to the controller.

13. The system of claim 1, wherein the temperature-sensing brassiere further comprises an inertial measurement unit:
arranged in the brassiere structure;
electrically coupled to the controller; and
configured to measure a posture of the user during the data collection period.

14. A system for evaluating breast cancer risk in a user comprising:
a temperature-sensing brassiere comprising:
a first sensor mesh comprising:
a first flexible mesh defining a mesh pattern and extending across a first breast region of the user;
a first set of temperature-sensing assemblies arranged at intersections in the first flexible mesh; and
for each temperature-sensing assembly in the first set of temperature-sensing assemblies, an electrical trace in the first mesh electrically coupling the temperature-sensing assembly to a controller;
a brassiere structure configured to locate the first sensor mesh across the first breast region of the user; and
the controller configured to:
sample each temperature-sensing assembly in the first set of temperature-sensing assemblies during a data collection period to generate a series of temperature data; and
store the series of temperature data.

15. The system of claim 14:
wherein the first flexible mesh comprises a first substrate comprising a first set of boustrophedonic segments arranged in the mesh pattern;
wherein each temperature-sensing assembly in the first set of temperature-sensing assemblies comprises:
a thermal contact fastened to the first substrate at an intersection in the first flexible mesh via a bore in the first substrate at the intersection in the first flexible mesh; and
a temperature sensor coupled to the first substrate at the intersection in the first flexible mesh and configured to detect the temperature of the thermal contact; and
wherein the brassiere structure is interposed between the first substrate and the thermal contact of each temperature-sensing assembly in the first set of temperature-sensing assemblies, wherein the thermal contact of each temperature-sensing assembly in the first set of temperature-sensing assemblies protrudes through the first substrate and the brassier structure to contact skin of the user proximal the first breast region of the user.

14. The system of claim 14:
wherein the first flexible mesh extends from immediately right of a sternum of the user across a right breast of the user to a right axillary region of the user and from below the right breast of the user across the right breast of the user to a right clavicular region of the user, when the temperature-sensing brassiere is worn by the user; and
wherein the temperature-sensing brassiere further comprises a second sensor mesh comprising:
a second flexible mesh defining the mesh pattern and extending from immediately left of a sternum of the user across a left breast of the user to a left axillary region of the user and from below the left breast of the user across the left breast of the user to a left clavicular region of the user, when the temperature-sensing brassiere is worn by the user;
a second set of temperature-sensing assemblies arranged at intersections in the second flexible mesh; and
for each temperature-sensing assembly in the second set of temperature-sensing assemblies, an electrical trace in the second mesh electrically coupling the temperature sensor to a controller.

17. A method for a breast cancer risk assessment comprising:
at a temperature-sensing brassiere comprising:
a first sensor mesh comprising:
a first flexible mesh defining a mesh pattern and extending across a first breast region of a user;
a first set of temperature-sensing assemblies arranged at intersections in the first flexible mesh; and
for each temperature-sensing assembly in the first set of temperature-sensing assemblies, an electrical trace in the first mesh electrically coupling the temperature-sensing assembly to a controller;
a second sensor mesh comprising:
a second flexible mesh defining the mesh pattern and extending across a second breast region of the user;
a second set of temperature-sensing assemblies arranged at intersections in the second flexible mesh; and
for each temperature-sensing assembly in the second set of temperature-sensing assemblies, an electrical trace in the second mesh electrically coupling the temperature-sensing assembly to a controller; and
a brassiere structure configured to locate the first sensor mesh across the first breast region of the user and locate the second sensor mesh across the second breast region of the user;
during a data collection period:
recording first breast temperature data from each temperature-sensing assembly in the first set of temperature-sensing assemblies and second breast temperature data from the second set of temperature-sensing assemblies; and
after the data collection period:
spatially interpolating the first breast temperature data and the second breast temperature data to generate a first breast temperature profile and a second breast temperature profile; and
estimating a breast cancer risk assessment for the user based on the first breast temperature data and the second breast temperature data.

18. The method of claim 17, further comprising, after the data collection period:
rendering the first breast temperature profile and the second breast temperature profile as a heat map representing the temperature of the first breast.

19. The method of claim 18, after the data collection period:
further comprising estimating a location of an abnormal mass in the first breast of the user or the second breast of the user; and
wherein rendering the first breast temperature profile and the second breast temperature profile further comprises rendering an indication of the location of the abnormal mass in the first breast temperature profile or the second breast temperature profile.

20. The method of claim 17 further comprising:
at the temperature-sensing brassiere further comprising an inertial measurement unit arranged in the brassiere structure;
during the data collection period, recording a series of inertial data from the inertial measurement unit; and
after the data collection period, in response to estimating a noncompliant posture of the user based on the series of inertial data, prompting the user to reinitiate the breast cancer risk assessment.
